(19) United States
(12) Plant Patent Application Publication
(10) Pub. No.: US 2003/0140389 P1
(43) Pub. Date: Jul. 24, 2003

(54) CHRYSANTHEMUM PLANT NAMED 'JMAY01'

(76) Inventor: James D. May, San Luis Obispo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/055,860

(22) Filed: Jan. 22, 2002

(51) Int. Cl. A01H 5/00

(52) U.S. Cl. PLT/287

(57) ABSTRACT

A new and distinct Chrysanthemum plant cultivar characterized by solitary, decorative flowers having a bright golden yellow coloration of the inner ray petals, gradually turning to a white coloration towards the outer ray petals, blooming consistently after 56 days of a 12/12 daylight/darkness cycle, having a free branching habit, and a consistent natural season flowering habit during the fall.

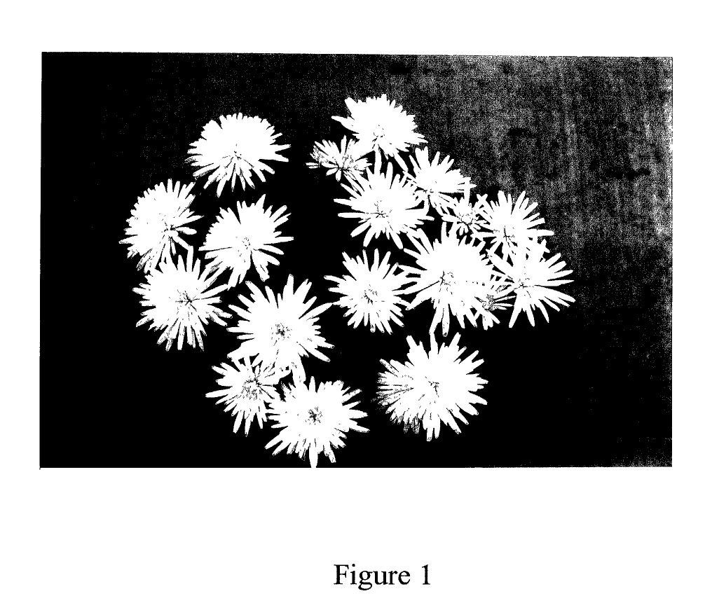

### BACKGROUND OF THE INVENTION

The present invention relates to a new and distinct cultivar of Chrysanthemum plant, botanically known as Dendranthema grandiflora and hereinafter referred to by the cultivar name ‘Sunburst Casper’.

The new cultivar is a two tone natural mutation of the variety ‘Casper’ (unpatented) discovered in a controlled greenhouse environment in San Luis Obispo, Calif.

Asexual reproduction of the new cultivar by apical tip cutting and meristem tissue culture was performed in San Luis Obispo, Calif., and has shown that the unique features of this new Chrysanthemum cultivar are stable and reproduce true to type on successive generations.

### SUMMARY OF THE INVENTION

The cultivar ‘Sunburst Casper’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘Sunburst Casper’. The new cultivar ‘Sunburst Casper’ naturally blooms in the fall season. The 10-12 cm solitary, decorative inflorescences have bright golden yellow inner ray petals of about 4.6 cm, which radiate out to light, almost white outer ray petals. Floral bloom occurs after about 56 days of a 12 hour light/12 hour darkness cycle. The color of the petals change as the flower ages: the outer ray petals fade to white with age, while the inner petals fade to a golden yellow with age. No seeds are produced. The plant is perennial, herbaceous, and has excellent disease resistance characteristics. The new cultivar ‘Sunburst Casper’ has a free branching semi-upright habit, growing to about 25-35 cm as a disbud pot mum. The fragrance of the herbage is strong and pungent, while the inflorescence has little to no fragrance. The mature leaves are about 8-10 cm in length by 6-8 cm in width, and have a lanceolate shape with an acute apex.

The new cultivar ‘Sunburst Casper’ can be distinguished as a new and distinct Chrysanthemum cultivar by its bright golden yellow coloration of inner ray petals radiating out to white outer ray petals.

Plants of the new cultivar are similar to plants of the parent cultivar, ‘Casper’ in most horticultural characteristics, differing from the parent cultivar ‘Casper’ primarily in bloom characteristics. Plants of the new cultivar can be distinguished from its parent cultivar ‘Casper’ by the bright golden yellow coloration of its inner ray petals and white coloration of its outer ray petals. The parent cultivar ‘Casper’, in contrast, has pale yellow inner ray petals.

Plants of the new cultivar are similar to the cultivar ‘Sunburst Spirit’ (U.S. Plant Pat. No. 4,476). The new cultivar can be distinguished from the cultivar ‘Sunburst Spirit’ by having a bright golden yellow coloration of its inner ray petals, as compared to the light yellow coloring of the inner ray petals of the ‘Sunburst Spirit’ cultivar.

### BRIEF DESCRIPTION OF THE PHOTOGRAPHS

The photograph shows a plant of the new cultivar which was grown in a 6½-inch container. Five cuttings were grown in the container, and the plant was grown under greenhouse conditions.

### DETAILED BOTANICAL DESCRIPTION

In the following description, color references are made to the Royal Horticultural Society Colour Chart except where general terms of ordinary dictionary significance are used. The following observations and measurements describe plants grown in a greenhouse in San Luis Obispo, Calif., in the month of September 2000. The growing conditions range from 60°0 to 65° F. at night to 70° to 85° F. during the day. Measurements and numerical values represent averages of typical flowering types.

Botanical classification: Dendranthema grandiflora cultivar ‘Sunburst Casper’

Commercial classification: Florist Chrysanthemum

Parentage: Natural mutation of the variety ‘Casper’ discovered in a controlled greenhouse environment.

### PROPAGATION

Time to Rooting: 3 to 5 days

Rooting habit: Basal swelling, callusing, and profuse root production just above base of cutting

### PLANT

Growth Habit: Semi-upright

Height: 25-35 cm as a disbud pot mum with 0-7 long days after pinch, but prior to short days and 2 applications of 2500 ppm B-9 sp

Growth rate: 2-3 cm per week after pinch

Branching Characteristics: Free branching

### FOLIAGE

Leaf:

Arrangement: Alternate

Average Length: Approximately 8-10 cm

Average Width: Approximately 6-8 cm

Shape of blade: Lanceolate

Apex: Acute

Base: Attenuate

Attachment: Attenuate to stem

Margin: Moderately to deeply lobed and slightly serrate

Texture of top surface: Canescent

Texture of bottom surface: Pubescent

Color:

young foliage upper side: Near yellow-green 147A

young foliage under side: Near green 137B

mature foliage upper side: Near green 139A

mature foliage under side: Near green 137B

venation upper side: Near green 138C

venation under side: Near green 138B

Petiole:

Average Length: Attenuate to stem—no pronounced petiole

### BLOOM

Inflorescence:

Flowering habit: Requires about 12 hours light/12 hours darkness for approximately 56 days to induce and develop blooms

Inflorescence form: Solitary

Bloom type: Decorative

Natural flowering season: Fall

Number of inflorescences per stem: One

Inflorescence diameter: About 10-12 cm

Inflorescence depth: About 2-3 cm

Inflorescence longevity on plant: Approximately 21-28 days

Persistence: Persistent

Ray Florets:

Appearance: Matte

Texture: Glabrous

Average Number per flower: Numerous

Shape: Oblanceolate

Margin: Entire

Tip/Apex: Obtuse to retuse

Base: Tapered

Aspect: Straight

Length: Approximately 4.6 cm

Width: Approximately 1.0 cm

Color:

upper surface at first opening: Near yellow 9B

upper surface at maturity: Near yellow 4C

upper surface at fading: Near white

under surface at first opening: Near yellow 9B

under surface at maturity: Near yellow 4C

under surface at fading: Near white

Peduncle:

Length: Approximately 0.8-1.2 cm

Strength: Strong

Color: Near green 138C

Habit: Erect

Surface: Pubescent

Inflorescence bud:

Length: Approximately 0.8-1.0 cm

Diameter: Approximately 1.2-1.4 cm

Form: Globular

Color: Near green 141B

Sepals:

Appearance: Matte

Texture: Pubescent

Number: Numerous—first set of sepals, then a single layer of ray florets followed by a second layer of several sepals thick, followed by inner petals

Shape: Ovate

Margin: Entire

Apex: Acute

Color:

upper side: Near green 131A

underside: Near green 139A

Reproductive Organs

Ray Florets:

Pistillate:

Number of pistils per flower: 1

Stigma shape: Linear

Stigma color: Near yellow-orange 17A

Style color: Near yellow-orange 17A

Style length: about 3-5 mm

Disc Florets:

Pistillate:

Number of pistils per flower: 1

Stigma shape: Linear

Style length: 3-5 mm

Staminate:

Number of stamens per flower: 5

Anther shape: Syngenesious

Anther color: Near yellow 8A at top, fading to near yellow 8D at base

Pollen color: Near yellow-orange 17A

Pollen quantity: Low

What is claimed is:

1. A new and distinct cultivar of Chrysanthemum plant named ‘Sunburst Casper’ as herein illustrated and described.
